(19) United States
(12) Patent Application Publication
(10) Pub. No.: US 2020/0061085 A1
(43) Pub. Date: Feb. 27, 2020

(54) COMPOSITION FOR TREATMENT, INHIBITION AND ATTENUATION OF MELANOMA VIRUS AND PREVENTION OF SKIN CANCERS

(71) Applicant: Frederick J. Sawaya, Farmington Hills, MI (US)

(72) Inventor: Frederick J. Sawaya, Farmington Hills, MI (US)

(21) Appl. No.: 16/109,719

(22) Filed: Aug. 22, 2018

(51) Int. Cl. A61K 31/616; A61K 31/194; A61K 31/19; A61K 33/04; A61P 35/00

(52) U.S. Cl. CPC A61K 31/616; A61K 31/194; A61P 35/00; A61K 33/04; A61K 31/19

(57) ABSTRACT

The embodiments herein relate to a therapeutically active composition for the treatment, inhibition or attenuation of a skin cancer or melanoma. The composition comprises an effective amount of a sulfur containing compound along with one or more pharmaceutically acceptable carriers or excipients. The sulfur containing compound impairs a disulfide bond of a plurality of virus. The sulfur containing compound is sodium thiosulfate (Na₂S₂O₃) (“STS”). The composition is given orally, intravenously, inhalation, intravesical, vaginal, rectal, sublingual, ophthalmic, or topical.

### BACKGROUND

### Technical Field of Invention

The present invention generally relates to a therapeutically active composition for prophylaxis, stoppage of inborn genetic trait that forms melanoma and inhibition or attenuation, treatment of local and metastatic organ and skin melanomas and skin cancers caused by viruses in human beings.

### DESCRIPTION OF RELATED ART

Cancer is a group of diseases involving abnormal cell growth with the potential to invade or spread to other parts of the body. These contrast with benign tumours, which do not spread to other parts of the body. Melanoma is an abnormal overgrowth of melanocyte cell in the skin which is subject to UV light. Melanoma is not the most common type of skin cancer, but it is the most serious type because it often spreads. Risk factors for melanoma include overexposure to the sun. Melanoma commonly mutates and metastasizes in white fair skinned red headed freckled people. The mortality rate in the metastatic condition is 75%.

Three pathogenic human viruses associated with skin neoplasms are human papilloma virus (HPV), Kaposi's sarcoma (KS)-associated herpes virus and human T-cell leukemia virus type 1. HPV was linked to squamous cell carcinoma (SCC) of the skin after its role in SCC of the cervix has been discovered.

Sulphur containing compounds have been proven for their effects on the impairment of S—S bonds and cause inhibition of the cancer growth but none can be used on herpes or viruses causing skin cancers.

The drug Bactrim is primarily antibacterial for the treatment of routine urinary tract infections. Bactrim contains a combination of sulfamethoxazole and trimethoprim. The Sulfamethoxazole and trimethoprim are both antibiotics that treat different types of infection caused by bacteria only.

Thus, there is a need to develop a new and safer compound or drug which has a quality of impairing the disulphide bonds in skin cancers and causing the mutation inhibition, impairment and death of cancer causing viruses, and prevention of skin cancers or melanomas.

The above mentioned shortcomings, disadvantages and problems are addressed herein, as detailed below.

### SUMMARY OF THE INVENTION

The primary object of the embodiments herein is to provide a composition for the treatment, inhibition and attenuation of cancers, including viruses causing cancers and the melanoma cancers themselves.

Another object of the embodiments herein is to provide a composition for the prevention of melanoma by causing disruption of arginine and cysteine formations leading to abnormal protein.

Yet another object of the embodiments herein is to provide a composition for redirection of the formation of normal amino acids vs abnormal ones.

Yet another object of the embodiments herein is to provide a composition comprising sulfur which has the capability of impairing the disulfide bonds and cause death of melanoma, and viruses causing melanoma such as herpes and HPV human papilloma viruses also includes cancer causing viruses.

Yet another object of the embodiments herein is to provide a composition that can be given orally, parenterally, IV and PO, and topically.

Yet another object of the embodiments herein is to provide a composition which is readily and cheaply available, easy to manufacture, and treats the viral infection.

According to an embodiment herein, a therapeutically active composition for prophylaxis (from birth and prenatal), inhibition of genetic inborn trait that forms melanoma, attenuation or treatment of local and metastatic organ and skin melanomas and skin cancers is provided. The composition comprises an effective amount of a sulfur containing compound along with one or more pharmaceutically acceptable catalysts, carriers or excipients, wherein the sulfur containing compound is sodium thiosulfate (Na2S2O3) (“STS”), and wherein the catalysts include acetyl salicylic acid (ASA), citric acid and apple cider vinegar, and wherein the one or more pharmaceutically acceptable carriers or excipients is water.

According to an embodiment herein, the melanoma includes malignant and non-malignant melanomas, basal cell carcinoma, squamous cell carcinoma, keratoses and actinic keratoses and general skin abnormalities.

According to an embodiment herein, the STS is used in atleast two forms, wherein the two forms are anhydrous form and pentahydrate form.

According to an embodiment herein, the composition is given orally, intravenously, inhalation, intravesical, vaginal, rectal, sublingual, ophthalmic, or topical.

According to an embodiment herein, the composition is given orally in the form of a tablet, powder, or a capsule.

According to an embodiment herein, the composition is given intravenously as an infusion of a solution containing STS.

According to an embodiment herein, the composition is topically administered in a form of a solution, cream, paste, or lotion containing STS.

According to an embodiment herein, an effective amount is between 1 mg to 2 g per kg of body weight per day of the treatment.

According to another embodiment herein, a method for prophylaxis, inhibition of genetic inborn trait of melanoma, attenuation or treatment of local and metastatic organ and skin melanomas and skin cancers is provided. The method comprises administering an effective amount of a sulfur containing compound along with one or more pharmaceutically acceptable catalysts, carriers or excipients, wherein the sulfur containing compound impairs a disulfide bond of a plurality of melanoma or virus precursor, wherein the sulfur containing compound is sodium thiosulfate (Na2S2O3) (“STS”), and wherein the catalysts include acetyl salicylic acid (ASA), citric acid and apple cider vinegar, and wherein the one or more pharmaceutically acceptable carriers or excipients is water.

According to an embodiment herein, the sodium thiosulfate (Na₂S₂O₃) (“STS”) biodegrades amino acids, polypeptide proteins and glycoproteins in the virus and releases H2S and SO2 as breakdown products.

According to an embodiment herein, the STS splits an S—S disulfide bond of a skin cancer and disrupts integrity of an item, organism or cancer or infection.

According to an embodiment herein, splitting the S—S disulfide bond and biodegrading the glycoprotein enables the STS to destroy any skin cancer.

According to an embodiment herein, the STS supplies exogenous electrons which confuses repair or formation of amino acids, peptides proteins or any S—S bond substance which is needed to create melanoma malignancies and any skin cancer or metastatic intracorporal SS bond repair.

According to an embodiment herein, the effective amount administered is between 1 mg to 2 g per kg of body weight per day of the treatment.

The embodiments herein provide a therapeutically active composition for the treatment, inhibition or attenuation of melanoma or skin cancer, malignant melanoma or any precursor virus leading to a melanoma. The composition comprises an effective amount of a sulfur containing compound along with one or more pharmaceutically acceptable carriers or excipients. The sulfur containing compound impairs a disulfide bond of a plurality of viruses. The plurality of viruses includes herpes and human papilloma virus (HPV). The sulfur containing compound is sodium thiosulfate Na₂S2O₃.

The composition is given orally in the form of a tablet, powder, or a capsule.

The composition is given intravenously as an infusion of a solution containing STS.

The composition is given by inhalation, intravesical, vaginal, rectal, sublingual, ophthalmic, or topical.

The composition is topically administered in the form of a solution, cream, paste, or lotion containing STS.

The one or more pharmaceutically acceptable carriers or excipients is water.

The amount is between 1 mg to 2 g per kg of body wt.

The sodium thiosulfate biodegrades amino acid polypeptide proteins and glycoproteins in a virus and releases H₂S and SO₂ as breakdown products. The STS splits an S—S disulfide bond of a virus and disrupts integrity of an item, organism or cancer or infection. The splitting of the S—S disulfide bond and biodegrading the cysteine or any amino acid or glycoprotein or peptide or lipids. STS destroys the melanoma precursor amino acid arginine and cysteine thereby stopping melanoma formation.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The other objects, features and advantages will occur to those skilled in the art from the following description of the preferred embodiment and the accompanying drawings in which:

FIG. 1 is a conceptual drawing showing the binding of virus on a human cell, according to an embodiment herein.

FIG. 2 shows a growth of melanoma onto a skin surface, according to an embodiment herein.

FIG. 3 shows a chemical formula of two cysteine molecules bonded together by a di-sulfide bond, according to an embodiment herein.

FIG. 4A shows Thiol-disulfide exchange showing the linear intermediate in which the charge is shared among the three sulfur atoms. The thiolate group (shown in red) attacks a sulfur atom (shown in blue) of the disulfide bond, displacing the other sulfur atom (shown in green) and forming a new disulfide bond, according to an embodiment herein.

FIG. 4B shows three different positions of the electrons replacement, according to an embodiment herein.

FIG. 4C shows a reaction wherein the sulfur dioxide and hydrogen sulphide reacts with the S—S bond, according to an embodiment herein.

### DETAILED DESCRIPTION OF THE DRAWINGS

In the following detailed description, a reference is made to the accompanying drawings that form a part hereof, and in which the specific embodiments that may be practiced is shown by way of illustration. The embodiments are described in sufficient detail to enable those skilled in the art to practice the embodiments and it is to be understood that the logical, mechanical and other changes may be made without departing from the scope of the embodiments. The following detailed description is therefore not to be taken in a limiting sense.

In the following detailed description Sodium Thiosulfate, STS and Na₂S₂O₃ are used interchangeably referring to sodium thiosulfate. The term S—S bond refers to disulfide bonds, H₂S refers to Hydrogen sulphide and SO₂ refers to sulfur dioxide. HS is a term referred to the active binding portion of H₂S.

The various embodiment of the present invention relates to the fields of microbiology and antimicrobial pharmacotherapy. More particularly the compositions and methods of the invention relate to action of the active sulfur present in sodium thiosulfate that binds metals or splits disulfide bonds in amino acids of viruses, peptides, melanoma and skin cancers or keratoses etc.

According to an embodiment herein, a composition for treatment, inhibition and attenuation of malignant melanoma, melanoma and other skin cancers or keratoses, etc, is provided. A therapeutically active composition for prophylaxis (from birth and prenatal) treatment, inhibition or attenuation of a skin cancer local or metastatic comprises an effective amount of a sulfur containing compound along with one or more pharmaceutically acceptable carriers or excipients, and one or more catalysts which are FDA approved.

According to an embodiment herein, the sulfur containing compound impairs a disulfide bond of a plurality of skin cancers. The plurality of skin cancers includes skin cancers, squamous cell carcinoma, basal cell carcinoma, actinic keratoses, melanoma and malignant melanoma.

According to an embodiment herein, the sulfur containing compound is sodium thiosulfate (Na₂S₂O₃) (“STS”). The IV form of STS is an FDA approved drug for cyanide poisoning, eclampsia and renal dialysis. The oral form of STS is in the early stages of successful clinical trials for varying diseases, specifically Alzheimer's and brain cell damages.

The composition is given orally in the form of a tablet, powder, or a capsule.

The composition is given intravenously as an infusion of a solution containing IV-injectable STS.

The composition is given by inhalation, intravesical, vaginal, rectal, sublingual, ophthalmic, or topical.

The composition is topically administered in the form of a solution, cream, paste, or lotion containing STS.

According to an embodiment herein, the catalyst includes acetylsalicylic acid (ASA) which helps to speed up the S—S-splitting by 200-2000 times.

According to an embodiment herein, vanilla extract is used as a preservative for STS and provides a flavor for administration to even children by making the sulfur-taste of the water more palatable.

According to an embodiment herein, the STS inhibits the production of Cysteine 24 which is a part of the antigenic peptide CDK4-R24C peptide ACDPHSGHFV (amino acids 23-32). These amino acids are abnormal precursors to the formation of the protein which is abnormal in melanoma. Consequently, the STS directly inhibits the formation of the building blocks of amino acids required to make melanoma amino acids and protein.

The STS is a proven antioxidant which favours the production of normal arginine and cysteine as opposed to the abnormal arginine in the production of abnormal cysteine to produce the abnormal protein of malignant melanoma.

The sulfur containing compound is effective against skin cancer and skin cancer forming viruses containing disulfide bonds, wherein the cancer cells (amino acids) containing disulfide bonds such as melanoma, malignant melanoma and basal and squamous cell cancers, actinic keratosis and premalignant lesions of skin, local or metastasized.

The sulfur containing compound attacks melanoma or any viral metal to disable or kill the melanoma by disabling the amino acid productive process including cancer virus.

The sulfur containing compound binds with the Polymerase Magnesium by a sulfur produced via STS, wherein said sulfur containing compound binds to any viral metal or heavy metal to disable or kill the virus.

The sulfur containing compound attacks the mucin component of a virus.

The sulfur containing compound releases heavy metal binding atoms and molecules.

According to another embodiment herein, a method for treatment, inhibition or attenuation of a skin cancer comprises administering an effective amount of a sulfur containing compound along with one or more pharmaceutically acceptable carriers or excipients, and/or catalysts, wherein the sulfur containing compound impairs a disulfide bond of a plurality of skin cancers and wherein said plurality of skin cancers includes melanoma, malignant melanoma, basal cell, squamous cell, and actinic keratosis or premalignant keratosis.

The sulfur containing compound is sodium thiosulfate (Na₂S₂O₃) (“STS”), wherein said sodium thiosulfate biodegrades amino acid polypeptide proteins and glycoproteins in a cancer cell and releases H₂S and SO₂ as breakdown products.

The STS splits an S—S disulfide bond of a skin cancer and disrupts integrity of an item, organism or cancer or infection.

The splitting the S—S disulfide bond and biodegrading the glycoprotein enables the STS to destroy any skin cancer. STS induces release of tetherin at the mitochondrial level and save anyone from skin cancers.

The STS supplies exogenous electrons which confuses repair &/or formation of amino acids, peptides proteins or any s-s bond substance which is needed to create melanoma malignancies and any skin cancer or metastatic intracorporal SS bond repair.

The composition provides a sulfur containing compound which is used in solid and liquid forms. The sulfur containing compound is sodium salt of sulfur. The sulfur containing compound is sodium thiosulfate (STS). The composition comprises sodium thiosulfate along with necessary additives. The composition includes catalysts specifically acetyl salicylic acid (ASA), citric acid and apple cider vinegar. The first two catalysts can be given IV, oral or topically or recto-vaginally, and the last vinegar is given topically.

The sulfur variation by products has the capability of impairing the disulfide bonds in viruses. In the human body, the sodium thiosulfate releases hydrogen sulfide, sulfur dioxide and S (sulphur atoms), and also forms SO₃-depending on the conditions. There are three sulfur atoms coming out of STS which eventually split S—S, digest protein amino acids, and stimulate the release of tetherin in the mitochondria, increases circulating GSTH which is glutathione stimulating hormone which inhibits many cancers from forming and inhibits viruses. SO₂ is also a by-product of STS under certain conditions in the body. SO₂ also can vividly break disulfide bonds in a virus or in vitro.

Sodium thiosulfate is a sulfur-based pharmaceutical agent and proven to split S—S bonds, open the blood-brain barrier of the human for cancer drugs to work and treats hypertension in pregnancy and reverse cyanide poisoning in all cases.

STS is a colourless crystalline compound that is more familiar as the pentahydrate, Na₂S₂O₃.5H₂O, an efflorescent, monoclinic crystalline substance also called sodium hyposulfite. Sodium thiosulfate is produced chiefly from liquid waste products of sodium sulfide or sulfur dye manufacture. In the laboratory, this salt is prepared by heating an aqueous solution of sodium sulfate with sulfur or by boiling aqueous sodium hydroxide and sulfuric acid according to this equation:

6NaOH+4S→2Na₂S+Na₂S₂O₃+3H₂O

The human liver, kidneys and testes produce thiosulfate reductase, which catalyzes STS producing hydrogen sulfide (H₂S), as follows Na₂S₂O₃ always supplies the S—S Split Atoms & Molecules. Hydrogen sulfide proof of the use delivery of H₂S from Na₂S₂O₃ Thiosulfate is a potential respiratory electron acceptor for bacteria which live in anoxic environments or at the anoxic/oxic interface. The ability to respire thiosulfate is conferred by the enzyme thiosulfate reductase which catalyzes the reaction. The body itself produces STS via cysteine but in limited quantities and original functions was the removal of trace heavy metals mercury lead arsenic from the body.

The self-production of cysteine of STS by the human body is likely why STS given exogenously for 90 years of cumulative uses related to renal, cyanide and eclampsia, have no fatalities—not one. Cysteine in the human is the parent of STS hence there is some created “relationship” between STS and human cells, since the STS never lyses and cysteine in the 90 years of use since the urines checked are not altered by. The ability to respire thiosulfate is conferred by the enzyme thiosulfate reductase which catalyzes the reaction.

S₂O₃²⁻+2H⁺+2e⁻→HS⁻+HSO₃⁻

SO₂ is also a by product of Na₂S₂O₃ under certain conditions in the body.

SO₂ also split disulfide bonds so that in using Na₂S₂O₃ two amino acid splitters are formed to kill melanoma or cancers or viruses, not just H₂S˜HS rather also SO₂ digest proteins peptides and amino acids. Furthermore, STS reacts with proteins, amino acids, and glycoproteins and produce hydrogen sulfide, H₂S and sulfur dioxide, SO₂, which then degrades glycoprotein, and H₂S & SO₂ continue by confusing the issue of sulfur replacement to the damaged finite disulfide bonds, by factoring infinite multiples of 6-8 electrons for every one sulfur damaged.

According to the embodiments herein, the safe sulfur containing compound is provided. The medically safe compound having the capability of splitting S—S bond is Na₂S₂O₃ (sodium thiosulfate, STS). At least two of the three sulfurs coming out from STS reactions splits S—S bonds, digest protein amino acids, of any cancer or foreign antigen using amino acides which includes everything in creation.

The self-regenerative properties of Na₂S₂O₃ to form Na₂S₄O₆, a tetrathionate is beneficial in self-regenerative available sulfur to destroy protein, bearing in mind that the human is immune to STS and is born with it as a metal eliminator and the reductase is inborn in liver, testes etc.

The catalyst Acetyl Salicylic Acid (ASA) increases the intravenous and oral or topical rate binding a metal by 200-2000 times tested in vitro, and the same rate of increase holds true for local and metastatic skin cancers even in the brain or eyeball.

Skin cancers like the viruses are susceptible to attack by STS, or by the sulfur in H₂S and SO₂. This is as follows: STS (SO2 and H2S) break these disulfide bonds, and literally digest amino acids in the cells of the skin cancers especially cysteine and arginine, which are the precursors to the abnormal protein leading to cancerous melanoma.

### Experimental Details

The glycoprotein (GP) or peptide has many amino acid variations. The glycoprotein and amino acids are similar to the beef broth. The study was conducted on beef broth solution.

The study using beef broth and sulfur (S) of Na₂S₂O₃ supports active sulfur binding of added copper and gas formation. The H₂S/S/SO₂ reactions were activated in the beef broth solutions by adding copper sulfate and Na₂S₂O₃ forming CuS. The sodium thiosulfate reacts with copper sulfate to form copper monosulfide, CuS, H₂S and SO₂. The H₂S alters amino acids in rat brains. The S, H₂S, SO₂ disrupt the lipid layer and is proven to disable S—S bonds and can cause protein degradation in an independent study.

In 1981, the typhirium outbreak study of Na₂S₂O₃ using beef broth and pectin in salmonella released H2S from the Na2S2O3 was given. In 2009 Photolytic Experiment proved splitting of S—S bonds via H2S. The damage of H₂S and SO₂ to bisulfide bonds is applicable to Ebola. In 2006 the Hampton report proved STS to cause protein degeneration. Other studies proved H₂S damaging to viruses, and cancer viruses and cancer tumors as well. STS chemically binds to many metals, including heavy metals.

Due to intrinsic proven production of metal-binding sulfur and production of available SO₂ and H₂S, and SO₃ permits multiple cancers or precancerous lesions to be stopped or never started. Using water in bottles of STS which are flavored with vanilla, which is also a preservative, can prevent genetic disruption or predilection of melanoma in childhood. Heavy metals such as mercury or lead or arsenic could be a factor in any disease and it is good to remove them.

Humans produce thiosulfate reductase in the liver, kidneys, and testes. It is also believed to be produced within the mitochondria of the human cell itself. When STS encounters this enzyme, the reduction product is hydrogen sulfide; the H₂S is the most aggressive form of binding sulfur on the earth which is human safe. STS is safe for all wildlife and it is also agriculturally safe while present in many food supplies/sources and is unrestricted in agriculture USDA Law 2009. In humans with cancer or skin infection, the sulfur in hydrogen sulfide and in STS attacks and degrades certain biochemical components of the virus, including: dissolution of the disulfide couplets present in certain glycoproteins, and S—S bonds are common denominators of most killer viruses and some cancers.

According to an embodiment of the present invention, the sodium thiosulfate can kill and impair cancer cell formation propagation metastases and the metastatic lesions intracorporeally themselves.

The cancer called “fibrosarcoma” and many or all solid cancer tumors, other cancers treatable by H2S/STS situations are: those having a probability in breaking ‘the disulfide bond’ between Cys⁴³⁷ and Cys⁵⁴². The Cys⁴³⁷ and Cys⁵⁴² are necessary for the secretion and activation of heparanase. The heparanase is a precursor to cancers. The overexpression of heparanase has been observed in many human tumor types, such as those in the head and neck pancreatic tumors, hepatocellular carcinoma, esophageal carcinoma and cultured human tumor cell lines.

H₂S is referenced in 2009 Photoelectric Experiment as breaking S—S bonds. No protein, no peptide no amino acid is immune to H₂S tenacious splitting—breaking of S—S bonds.

The sulfur containing compound impairs a disulfide bond of a plurality of skin cancers and melanoma local and metastatic. STS destroys S—S bonds of precursor abnormal cysteines and arginine leading to the mutant protein of melanoma. STS disrupts and lyses the S—S bonds of CDKN2A, CDKN2B and CDK4 genes involved in melanoma formation. Sulfur preferentially kills herpes of the mouth in a few minutes. STS creates a signalling molecule H2S in human safe format which H2S creates GSTH. STS increases the production of glutathione stimulating hormone which is proven to guide cells in a normal direction vs an abnormal direction. STS creates HS radicals which enter the mitochondria of any cell and can create a signalling molecule effect warning of an intruder.

Teleology means things end up in the direction they were intended to be for go or normal, nature tends toward the normal. Be it an abnormal virus, protein, cancer or precursor of any disease. Diseases do not have normal amino acids they all have code numbers and as in gastric cancer, and oral cancer are subject to the premise that STS-H2S can signal and single out an abnormal protein as in the herpes experiment as noted in the normal cell sparing article. The HS enters the mitochondria in a skin cell or any cell and decides if it is normal and also in the kidney simultaneously increases the production of KLOTHO the gene which controls aging. In so doing the signalling there is an increased production of tetherin which is the guardian alarm warning system of the body. STS˜H2S binds heavy and some non-heavy metals which are causing imbalance in amino add production or a proclivity to cancer abnormalities and remove heavy metal cancer precursor.

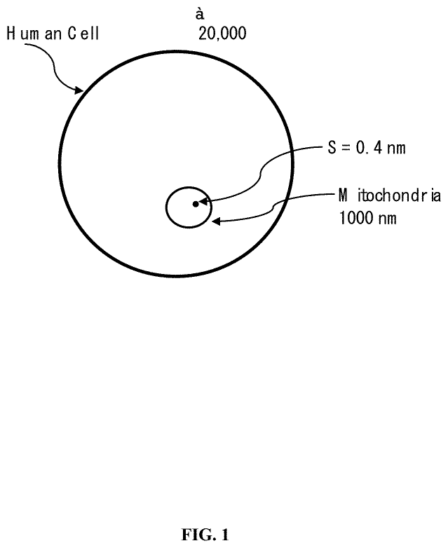

FIG. 1 is a conceptual drawing showing the binding of virus on a human cell, according to an embodiment herein. With respect to FIG. 1, the SO₂ is 0.3 μm, H₂S is 0.3 μm while sodium thiosulfate is 0.8 μm. The sulphur atom is shown as a back dot inside the mitochondria. The size of the mitochondria is 1000 nm while the Sulfur atom is 0.3-0.4 nm. The sulphur atom is small enough to safely enter mitochondria which is 1000 nm in size and that triggers tetherin release which signals the entire body of the cancer intruder or any infection. Thus, the sulphur atom enters the cell being attacked and destroys S—S bonds and digests amino acids peptides and proteins and glycoproteins also.

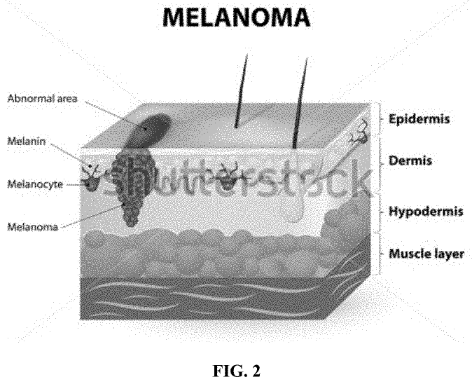

FIG. 2 shows a growth of melanoma onto a skin surface, according to an embodiment herein. With respect to FIG. 2, the melanocyte cells outgrow and leads to the formation of extra growth on the skin. This abnormal growth is known as melanoma or skin cancer.

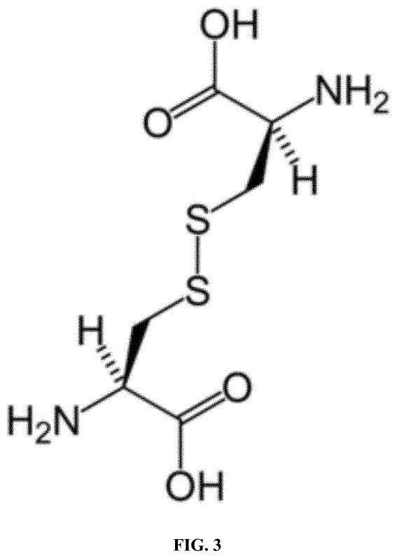

FIG. 3 shows a chemical formula of two cysteine molecules bonded together by a di-sulfide bond, according to an embodiment herein. With respect to FIG. 3, the interference of arginine onto normal cysteine alters the cysteine to promote abnormal protein which is the active agent of malignant melanoma. The STS attacks this S—S bond. The trick of disruption, and biodegradation of a melanoma or skin cancer protein matrix or a microbe or other entity is that when a S—S bond breaks, it has a replacement waiting a sulfur. However, if competitive Sulfur atoms each with 8 electrons are crowding the delivery shelf, then the electron matching process is another ball game of protracted time and/or missing and non-replaced “S” parts. The confusion of choice in replacement of the broken-missing bond, delays repair and leave a hole and vulnerability hopefully to further degradation. It is a treatment by outnumbering the cancers electrons with the Na₂S₂O₃'s electrons and mathematically it can outnumber the cells and tumors themselves.

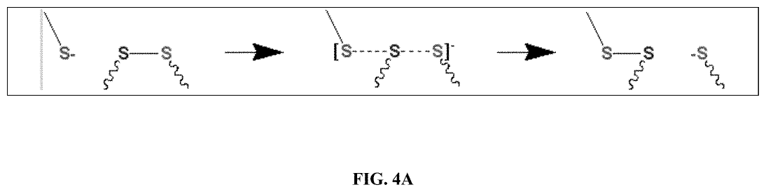

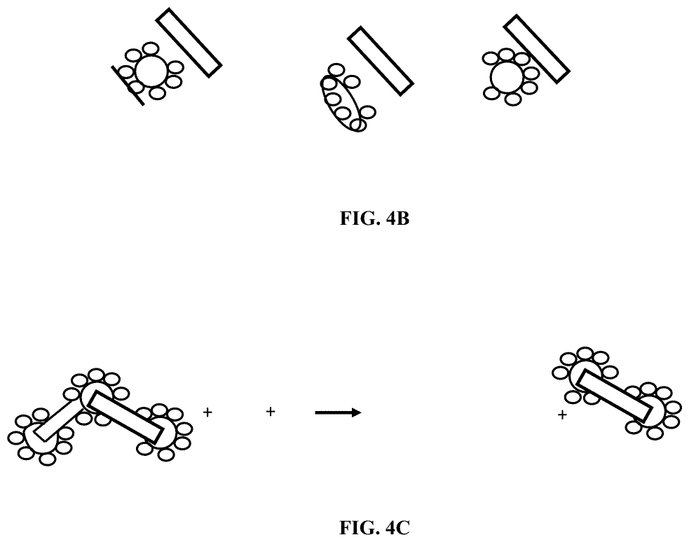

Referring now to FIG. 4A, thiol-disulfide exchange showing the linear intermediate in which the charge is shared among the three sulfur atoms. The thiolate group (shown in red) attacks a sulfur atom (shown in blue) of the disulfide bond, displacing the other sulfur atom (shown in green) and forming a new disulfide. A disulfide bond, also called an S—S bond, or disulfide bridge, is a covalent bond derived from two thiol groups. In biochemistry, the terminology R—S—S—R connectivity is commonly used to describe the overall linkages. The most common way of creating this bond is by the oxidation of sulfhydryl groups.

FIG. 4B shows the various degrees for the sulfur atom replacement in a disulfide bond, according to an embodiment herein. With respect to FIG. 4B, the ideal replacement for a horizontal loss is a horizontal sulfur. If this horizontal replacement does not take place, then a mutation happens. the cancer has a lunch box tool kit which has the exact one which was lost but if interference occurs, the “Brain” of the damaged S═S bond can't figure out which Sulfur to accept and maybe already gets the S from H₂S which is not aligned further the electrons from each sulfur are 8. So, multiples of S outnumber the sulfur atom replacement and confuse the issue. The S of H₂S always chooses to destroy a bond, and not to bond at all to the disulfide bond. The missing sulfur can be replaced in multiple degrees such as vertical, horizontal and tangential etc, according to the formula x/360° alignment. The melanoma cancer has a reserve Sulfur bank, as do most Disulfide bond creatures have, but the sulphur to be replaced from the melanoma cancer ‘tool box” would have to be the exact symmetry as the missing one, and ordinarily the replacement will happen with some positioning or juggling; however, delays or interferences (as caused by STS and S, SO₂, H₂S will create mutations as described in the in vitro studies of S—S bonding in the literature. The replacement at vertical and tangential degrees will not work if the missing Sulfur was horizontal for the sake of discussion or will take some readjusting and time to fix the right position which can result in a mutation and death of the melanoma, skin cancer or skin cancer virus.

Now referring to FIG. 4C, the reaction of sodium thiosulfate in presence of sodium thiosulfate reductase (STR) gives H₂S and SO₂. H₂S is relentless S—S bond splitter and so is SO₂. The enzyme sodium thiosulfate reductase is present in humans, white rats and mammals. The theory is to bombard the melanoma cancer repair shop with misaligned sulfurs which selectively only break sulfur bonds disulfide (bi).

One sulphur is never alone and can be 6+ at a time creating 48 electrons from one composite. The repair of split S—S bonds is not simple. The complexity of S replacement as referring to electron alignment and the multiplicity of S and concomitant electrons to overwhelm the mechanics of the virus play a role in damaging the melanoma cancer. The use of a multiple Na₂S₂O₃ which has Sulfur tetrathionate forming sulfurs each along with 4 modes of sulfur and 6-8 electrons per sulfur poses a serious unsolvable mathematical problem to any S—S bond repair virus or microbe, etc. The humans are born with Na₂S₂O₃ and bonds we are not broken by it. If the melanoma cancer or any item has a repair slot of sulfurs, any major change lower or higher will alter the mechanic so lower number are not enough to repair, too many numbers such as 10 sulfurs are hanging around from a few Na₂S₂O₃ molecules that is 80 electrons. In all sorts of arrangements and positions to be adjusted for and the cancer or repairee cannot do it adequately. It involves the Sulfur and its 6-8 electrons positionally on Na₂S₂O₃ pose another variable in having 4 sulfurs forms available. Na₂S₂O₃ does so by adding multiple sulfur atoms i.e. 6-8 electrons/sulfur atom as variables unwanted and unexpected by the virus repair machine.

The repair in a split amino acid bond is dependent on electron alignment. Each sulfur has 8 electrons. So, the missing slot needs just a set up to receive 8 electrons around the new Sulfur. If there is misalignment of electrons in the replacing sulfur in the broken S—S bond then hesitation, confusion and delays can occur. The Na₂S₂O₃ poses another variable in having 4 sulfurs forms available. Each sulfur has 8 electrons. So the missing slot needs just one sulfur to align correctly, not angled not backwards, not tangential, but figuratively “exactly flush” i.e. the atom has to be horizontal. But any selected angle would likely be the best one comprising vertical, horizontal and tangential.

The Sulfur replacement of a broken-disulfide bond, S—S includes electron alignment as well. Each Sulfur has 6-8 electrons. Normally a split S—S is replaced by the S located in amino acids inventory. But the replacement has to be just like fitting 8 holes on a wheel fitting an 8-bolt axle. If the wheel is angled or tangential or horizontal in the wrong place electrons don't match. As if they came off the amino acid rack ready to install. You can't just throw a sulfur in an empty hole. There are 6 electrons to fit also. If the variables exist as below like (A) & (B) then the empty slot for sulfur is inconvenienced & confused (1) too many sulfurs or (2) too many electrons. The use of a multiple Na₂S₂O₃ which has Sulfur tetrathionate forming sulfurs each along with 4 modes of sulfur and 8 electrons per sulfur poses a serious unsolvable mathematical problem to any S—S bond repair virus or microbe etc. The humans are born with STS and S—S bonds but the S—S bonds in humans are immune to STS.

TABLE 1
Below shows the replacement of S Atoms

| | | Middle |
|---|---|---|
| Right-FIG. 4B | Left-FIG. 4B | use FIG. # 4B |
| A sulfur missing (assume | A normal sulfur | 3 positions of S′ receptor |
| the lost sulfur was | horizontally repaired | for S can be in just one |
| horizontally aligned. | and replaced | of 3 positions. The donor |
| Shown in 4B | | (S) (8e) must match the |
| | | receptor |

If any skin cancer or melanoma item has a repair slot of sulfurs. Any major change lower or higher alters the mechanics. Too low means not enough to repair, too many means if 10 sulfurs are hanging around from a few Na₂S₂O₃ molecules that is 80 electrons. In all sorts of arrangements and positions to be adjusted for and the Virus or repairee cannot do it adequately.

The size of the sodium thiosulfate atom is 0.8 nm. The size of sodium atom is 0.4 nm while the size of sulfur atom is 0.4 nm. The size of mitochondria is 1000 nm.

H₂S═H₂S═H₂S=^{˜}<0.300 nm (1)

SO₂═SO₂═SO₂=^{˜}<0.300 nm (2)

With respect to (1) and (2), the sizes of H2S and SO2 are approximately the same.

The sodium thiosulfate (STS) by-products combine to eventually reform STS after a series of simple steps. For example, SO2+SO2 will eventually reform STS after a series of simple steps.

Thus, the present invention provides a composition for the treatment of viral infections. The present invention provides a sodium containing compound for the inhibition of the viral infection. The present invention provides a therapeutic composition for the treatment, attenuation and inhibition of viruses in human body. The sodium containing compound inhibits the disulfide bonds in viruses and cancers and cause their death. The composition is easy to prepare and administer.

The monetary issue of the oral form allows affordable health and avoidance of death using the very inexpensive oral form makes the use of a vaccine possibly secondary not obsolete. No reported Fatalities in 22 years of IV use for Cyanide and Pregnancy-Eclampsia.

Many melanoma or skin cancer prone people vs-as opposed to a vaccine one or two at best, or the mmr. For the most part, much of the expense of creating a vaccine may be spared by using an IV drug 100% safe for pregnant wife and unborn child.

The Oral ingestion of STS as a food preservative or salt additive or food supplement as done in Salt Packets, seems logical and safe for sure as shown in 2009 Federal Ruling USDA “Of Total Exemption of STS” from a restriction as an agricultural additive to fertilizer.

The trick of disruption, and biodegradation of a viral protein matrix or a microbe or other entity is that when a S—S bond breaks, it has a replacement waiting a sulfur. However, if competitive Sulfur atoms each with 8 electrons are crowding the delivery shelf, then the electron matching process is another ball game of protracted time and/or missing and non-replaced “S” parts the confusion of choice in replacement of the broken-missing bond, will delay repair and leave a hole and vulnerability hopefully to further degradation.

STS safely biodegrades amino acids polypeptides proteins and glycoproteins in any virus by mean of itself, H₂S and SO₂ its main breakdown products. STS splits the bonds of any “any” S—S disulfide bond (cancer or other) and disrupt the integrity of that item, organism, or cancer or infection. The splitting the S—S bonds and biodegrading the glycoprotein of Na2S2O3 active ingredient H2S & SO2 which destroys and biodegrades all proteins.

The sulphur electron competitive confusion entity factor wherein Extra unneeded electrons and sulphur disrupt the repair process of other skin cancers and skin cancer viruses and cancers.

It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the claims.

I claim:

1. A therapeutically active composition for prophylaxis (from birth and prenatal), inhibition of genetic inborn trait that forms melanoma, attenuation or treatment of local and metastatic organ and skin melanomas and skin cancers, wherein the composition comprises:
an effective amount of a sulfur containing compound along with one or more pharmaceutically acceptable catalysts, carriers or excipients;
wherein the sulfur containing compound is sodium thiosulfate (Na₂S₂O₃) (“STS”); and
wherein the catalysts include acetyl salicylic acid (ASA), citric acid and apple cider vinegar; and
wherein the one or more pharmaceutically acceptable carriers or excipients is water.

2. The composition according to claim 1, wherein the melanoma includes malignant and non-malignant melanomas, basal cell carcinoma, squamous cell carcinoma, keratoses and actinic keratoses and general skin abnormalities.

3. The composition according to claim 1, wherein the STS is used in at least two forms, wherein the two forms are anhydrous form and pentahydrate form.

4. The composition according to claim 1, wherein the composition is given orally, intravenously, inhalation, intravesical, vaginal, rectal, sublingual, ophthalmic, or topical.

5. The composition according to claim 1, wherein the composition is given orally in the form of a tablet, powder, or a capsule.

6. The composition according to claim 1, wherein the composition is given intravenously as an infusion of a solution containing STS.

7. The composition according to claim 1, wherein the composition is topically administered in a form of a solution, cream, paste, or lotion containing STS.

8. The composition according to claim 1, wherein an effective amount is between 1 mg to 2 g per kg of body weight per day of the treatment.

9. A method for prophylaxis, inhibition of genetic inborn trait of melanoma, attenuation or treatment of local and metastatic organ and skin melanomas and skin cancers, comprises:
administering an effective amount of a sulfur containing compound along with one or more pharmaceutically acceptable catalysts, carriers or excipients, wherein the sulfur containing compound impairs a disulfide bond of a plurality of melanoma or virus precursor,
wherein the sulfur containing compound is sodium thiosulfate (Na₂S₂O₃) (“STS”); and
wherein the catalysts include acetyl salicylic acid (ASA), citric acid and apple cider vinegar; and
wherein the one or more pharmaceutically acceptable carriers or excipients is water.

10. The method as claimed in claim 9, wherein the sodium thiosulfate (Na₂S₂O₃) (“STS”) biodegrades amino acids, polypeptide proteins and glycoproteins in the virus and releases H₂S and SO₂ as breakdown products.

11. The method as claimed in claim 9, wherein STS splits an S—S disulfide bond of a skin cancer and disrupts integrity of an item, organism or cancer or infection.

12. The method as claimed in claim 9, wherein splitting the S—S disulfide bond and biodegrading the glycoprotein enables the STS to destroy any skin cancer.

13. The method as claimed in claim 9, wherein the STS supplies exogenous electrons which confuses repair or formation of amino acids, peptides proteins or any S—S bond substance which is needed to create melanoma malignancies and any skin cancer or metastatic intracorporal SS bond repair.

14. The method according to claim 9, wherein the effective amount administered is between 1 mg to 2 g per kg of body weight per day of the treatment.
